(19) United States
(12) Patent Application Publication
(10) Pub. No.: US 2026/0026828 A1
(43) Pub. Date: Jan. 29, 2026

(54) RETRIEVAL DEVICES AND RELATED METHODS

(71) Applicant: Boston Scientific Scimed, Inc., Maple Grove, MN (US)

(72) Inventors: John SUBASIC, Sutton, MA (US); Ali ZIEGLER, Brookline, MA (US)

(73) Assignee: Boston Scientific Scimed, Inc., Maple Grove, MN (US)

(21) Appl. No.: 19/273,449

(22) Filed: Jul. 18, 2025

Related U.S. Application Data
(60) Provisional application No. 63/674,400, filed on Jul. 23, 2024.

(51) Int. Cl. A61B 17/221; A61B 17/00

(52) U.S. Cl. CPC A61B 17/221; A61B 2017/00867; A61B 2017/2215

(57) ABSTRACT

Medical devices, systems, and related methods useful for removing objects from patient anatomy during a medical procedure are described. The medical device may include a sheath defining a lumen, and a retrieval device slidably disposed within the lumen. The retrieval device may include a shaft including an expandable frame and a cover coupled to the frame. The retrieval device may move between a collapsed configuration while the frame is within the sheath and an expanded configuration while the frame is outside the sheath. The retrieval device may be configured to receive a laser fiber therein, such that the laser fiber may be used to break apart objects while within the retrieval device to facilitate removal of objects from patient anatomy. The cover may be laser-resistant, porous, and configured to prevent passage of object particles therethrough.

### CROSS-REFERENCE TO RELATED APPLICATIONS

This application claims the benefit of priority to U.S. Provisional Application No. 63/674,400, filed on Jul. 23, 2024, which is incorporated by reference herein in its entirety.

### TECHNICAL FIELD

Aspects of the present disclosure relate generally to medical devices and actuation mechanisms for medical devices. More specifically, the present disclosure relates to medical systems and devices with expandable frames and covers to facilitate lasing a captured object during medical procedures.

### BACKGROUND

During a medical procedure, medical professionals often use a basket or other grasper to engage kidney stones or other objects within patient anatomy to remove the objects. In the case of kidney stones, a laser fiber or similar instrument may be used to break apart the object for removal. However, this energy exposure may cause retropulsion of the object(s) relative to the retrieval device, which can complicate removal of the object fragments and may introduce risks to a patient during the procedure.

### SUMMARY

The present disclosure relates to, among other things, medical devices and systems for removing objects from body lumens and/or other body passageways, and related methods of use. Each aspect disclosed herein may include one or more features described in connection with any other disclosed aspect.

According to some aspects, the present disclosure includes a medical device including a sheath defining a lumen, and a retrieval device slidably disposed within the lumen. The retrieval device may include a shaft including an expandable frame at a distal end of the shaft, and a cover coupled to the frame. The retrieval device may be movable between a collapsed configuration while the frame is within the sheath and an expanded configuration while the frame is outside the sheath. The cover may be porous and configured to prevent passage of particles greater than 5 mm in diameter therethrough. Each of the frame and the cover may include a laser-resistant material. In some examples, the sheath has a diameter ranging from about 2 mm to about 3 mm.

According to some aspects, the retrieval device may be configured to allow passage of a laser fiber into a volume defined by the frame and the cover in the expanded configuration. The frame may be self-expandable. For example, the frame may comprise Nitinol. The frame may include a plurality of struts, e.g., at least three struts or at least four struts. In some examples, the cover may be coupled to each strut of the plurality of struts. In some examples, the plurality of struts of the frame may include at least one strut coupled to the cover and at least one strut unattached to the cover. The cover may include a laser-resistant polymer. The laser-resistant polymer may comprise, for example, polypropylene, polyethylene, or a combination thereof. In some examples, the laser-resistant polymer of the cover comprises high-density polyethylene, ultra-high-molecular-weight polyethylene, cross-linked polyethylene, or a combination thereof. The cover may include pores that are less than 1 mm in size. The cover may have a rolled configuration in the collapsed configuration of the retrieval device, the cover being movable between the rolled configuration and an unrolled configuration in the expanded configuration of the retrieval device. The cover may be movable between the rolled configuration and the unrolled configuration by rotating the shaft relative to the sheath. According to some aspects, a portion of the cover may be coupled to the sheath.

The present disclosure also includes a medical device comprising a retrieval device that includes a shaft including an expandable frame at a distal end of the shaft, and a cover coupled to the frame and movable between a rolled configuration and an unrolled configuration. Optionally, the medical device may further comprise a sheath, wherein the retrieval device is slidable disposed within a lumen of the sheath. The frame of the retrieval device may include a plurality of struts. The cover may include a laser-resistant material that is porous and configured to prevent passage of particles greater than 5 mm in diameter therethrough. In the rolled configuration, the cover may be coupled to fewer than all of the struts of the frame, and in the unrolled configuration, the cover may be coupled to all of the struts of the frame.

The present disclosure also includes methods of using the medical devices described above and elsewhere herein. The method may include positioning a medical device proximate a target site within a body lumen of a patient, wherein the medical device includes a sheath that defines a lumen and a retrieval device slidably disposed within the lumen, the retrieval device including an expandable frame at a distal end of the shaft and a cover coupled to the frame. The method may further include moving the retrieval device distal to the sheath such that the frame transitions from a collapsed configuration within the sheath to an expanded configuration outside the sheath, and capturing an object at the target site within a volume defined by the frame and the cover in the expanded configuration of the retrieval device. The method may further include inserting a laser fiber through the sheath and into the volume, and operating the laser fiber to break apart the object, where the cover includes a laser-resistant material that is porous, such that the cover prevents passage of particles greater than 5 mm in diameter therethrough. According to some aspects, operating the laser fiber generates dust particles less than or equal to 1 mm in size that pass through the cover.

### BRIEF DESCRIPTION OF THE FIGURES

The accompanying drawings, which are incorporated in and constitute a part of this application, illustrate aspects of this disclosure and together with the description, serve to explain the principles of this disclosure.

FIGS. 1A-1C depict an exemplary medical device including a retrieval device and a sheath, according to some aspects of the present disclosure.

FIGS. 2A-2C depict another exemplary retrieval device, according to some aspects of the present disclosure.

FIGS. 3A-3D show the retrieval device of FIGS. 2A-2C during different stage of deployment of the cover of the retrieval device.

### DETAILED DESCRIPTION

Both the foregoing general description and the following detailed description are exemplary and explanatory only and are not restrictive of the features, as claimed. As used herein, the terms “comprises,” “comprising,” “including,” “includes,” “having,” “has,” or any other variation thereof, are intended to cover a non-exclusive inclusion, such that a process, method, article, or apparatus that comprises a list of elements does not include only those elements, but may include other elements not expressly listed or inherent to such process, method, article, or apparatus. The term “exemplary” is used in the sense of “example,” rather than “ideal.” Relative terms such as “about,” “substantially,” and “approximately,” etc., are used to indicate a possible variation of +10% of the stated numeric value or range. The terms “proximal” and “distal” are used herein to refer to the relative positions of the components of exemplary medical devices. As used herein, “proximal” refers to a position relatively closer to the exterior of the body or closer to an operator using the medical device (see proximal “P” and distal “D” directional arrows in the figures). In contrast, “distal” refers to a position relatively further away from the operator using the medical device, or closer to the interior of the body.

Aspects of the present disclosure are described herein with reference to exemplary medical devices, systems, and methods that may be useful for removing objects from the body, such as removing a kidney stone from the urinary tract. For instance, exemplary medical devices herein may include a retrieval device slidably disposed within a lumen configured to capture an object, e.g., a kidney stone, from patient anatomy. The retrieval device may be configured to receive a laser fiber therein, such that the laser fiber may be used to break apart the object while within the retrieval device to facilitate removal of the object from the patient.

References to a particular type of procedure, such as a urology procedure; medical device, such as a basket or grasper; organ, such as a kidney; and/or object, such as a stone or stone fragment, are provided for convenience and not intended to limit this disclosure. Accordingly, analogous medical devices or systems may utilize one or more aspects of exemplary medical devices, systems, and methods described herein. Reference is now made in detail to examples to help illustrate aspects of the present disclosure through the accompanying drawings. Wherever possible, the same reference numbers shall be used throughout the drawings to refer to the same or like parts.

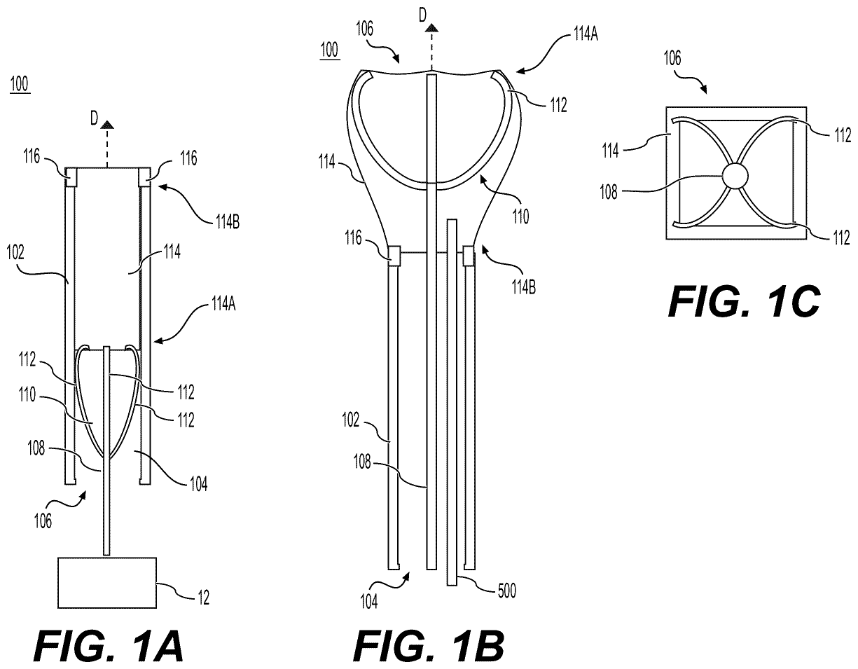

FIGS. 1A-1C illustrate an exemplary medical device 100 according to some aspects of this disclosure, medical device 100 including a sheath 102 and a retrieval device 106 with a shaft 108 coupled to a handle 12. The sheath 102 may be part of any suitable device configured to allow a user (e.g., medical professional) to access internal areas of a subject's body, e.g., to perform medical diagnoses and/or treatments on the subject. For example, the sheath 102 may be a sheath of a ureteroscope, an endoscope, a hysteroscope, a bronchoscope, a cystoscope, or other scope or similar medical device, wherein the retrieval device 106 is insertable into, and slidable along, a working channel that defines the sheath 102.

As shown and discussed herein, the retrieval device 106 includes shaft 108 slidably disposed in a lumen 104 of the sheath 102, a frame 110 at a distal end of the shaft 108 and a cover 114 coupled to the frame 110. The shaft 108 is coupled to the handle 12, such that the retrieval device 106 is movable along the lumen 104 and relative to the sheath 102 via the handle 12. The frame 110 may be expandable, e.g., self-expandable. For example, the retrieval device 106 may be movable between a collapsed configuration while the frame 110 is constrained within the sheath 102 (e.g., as shown in FIG. 1A), and an expanded configuration while the frame 110 is unconstrained by outside the sheath 102, distal to a distal end of the sheath 102 (e.g., as shown in FIG. 1B). In the expanded configuration, the frame 110 expands radially outward to form a cavity or volume proximate the distal end of the sheath 102 capable of enclosing one or more objects to be removed from the body.

The handle 12 may be used to control movement of the shaft 108 along the lumen 104 of the sheath 102. The handle 12 may include a grip portion having any suitable shape to be grasped by a user. Optionally, the handle 12 may include an actuator, such as a button, a finger ring, a switch (e.g., toggle switch), etc. configured to deploy the frame 110 once the frame 110 is advanced beyond the distal end of the sheath 102. The frame 110 may include a self-expandable material such as Nitinol, which may allow the retrieval device 106 to self-expand while positioned outside the sheath 102. The retrieval device 106 may include a volume defined by the frame 110 in the expanded configuration. The frame 110 includes one or more struts 112 extending radially outward relative to a central longitudinal axis of the shaft 108. The struts 112 of the frame 110 are configured to radially expand between the collapsed configuration while positioned within the sheath 102 (FIG. 1A), and the expanded configuration while positioned outside of the sheath 102 (FIG. 1B). In the example shown in FIGS. 1A-1C, the frame 110 includes four struts having proximal portions coupled to or integral with the shaft 108 and distal portions coupled to the cover 114. It should be understood that the retrieval device 106 may include more or fewer struts 112 defining the frame 110 than the example of FIGS. 1A-1C.

In some aspects, the struts 112 may be evenly spaced apart from each other, however other configurations may be used. In some examples, each of the struts 112 includes a self-expandable, elongate member having a proximal portion coupled to or integral with the shaft 108, and a distal portion opposite the proximal portion. Only one strut 112, several struts 112, or all struts 112 may be coupled to the cover 114. In some examples, at least one strut 112 includes a distal portion attached to the cover 114. In some examples, at least one strut 112 is attached to the cover 114 and at least one strut 112 is unattached to the cover 114. In some examples, the distal portions of two or more struts 112 are coupled together.

The retrieval device 106 may be configured to permit passage of a medical instrument such as a laser fiber 500 inside the frame 110 when expanded. For example, a laser fiber 500 may be introduced into the lumen 104 of the sheath 102 and moved distally so that the laser fiber 500 enters a volume defined by the frame 110 and the cover 114 in the expanded configuration (FIG. 1B). In some examples, the struts 112 are spaced apart with sufficient clearance to permit a laser fiber 500 to pass through an opening between adjacent struts 112 to enter the volume defined by the frame 110 and the cover 114.

In some examples, the frame 110 and/or the cover 114 may comprise laser-resistant materials to facilitate use of the laser fiber 500 within the retrieval device 106. For example, the cover 114 may comprise one or more laser-resistant polymer materials. Exemplary materials suitable for the cover 114 may include, but are not limited to, polypropylene, polyethylene, and combinations thereof. In some examples, the cover comprises high-density polyethylene (HDPE), ultra-high-molecular-weight polyethylene (UHMWPE), cross-linked polyethylene (XLPE), or a combination thereof.

The cover 114 may be configured to allow passage of smaller particles therethrough while retaining larger fragments within the retrieval device 106 for removal. In the case of kidney stones, for example, laser entry may be used during lithotripsy to reduce the size of a stone for removal from the body. Small dust particles generated from the procedure may be sufficiently small to be removed by irrigation and suction through the sheath 102 or flushed naturally through the urinary system with low risk to the patient. Larger fragments may be retained within the retrieval device 106 for removal by withdrawing the retrieval device 106 from the patient, e.g., pulling the retrieval device proximally through the lumen 104 of the sheath 102. The cover 114 may comprise a mesh or otherwise be porous with sizes of the pores selected to permit passage of small particles therethrough, e.g., dust particles, while preventing passage of larger fragments, e.g., fragments of a kidney stone to be removed from a patient. In some examples, the cover 114 may have a pore size less than or equal 5 mm, e.g., less than or equal to 4 mm, less than or equal to 3 mm, less than or equal to 2 mm or less than or equal to 1 mm. For example, the cover 114 may have a pore size of about 1 mm, such that the cover 114 prevents passage of particles/fragments greater than 1 mm in diameter therethrough while allowing smaller particles to exit cover 114 and retrieval device 106. The cover 114 may be coupled to, and movable relative to, the sheath 102. In some examples, translating the shaft 108 through the sheath 102 causes the cover 114 to invert due to coupling between the frame 110 and the cover 114.

The cover 114 may include at least one portion coupled to the frame 110, and at least one portion coupled to the sheath 102. As shown in the example of FIGS. 1A-1C, the cover 114 includes a first portion 114A (e.g., a first end) coupled to the frame 110, and a second portion 114B (e.g., a second opposite end) coupled to a distal portion of the sheath 102. Advancing the shaft 108 distally relative to the sheath 102 may cause the first portion 114A of the cover 114 to move relative to the second portion 114B, pulling the cover 114 outside of the sheath 102 as the frame 110 exits the lumen 104.

According to some aspects, the cover 114 may be coupled to the frame 110 via engagement elements. In some examples, the engagement elements may provide for portions of the cover 114 to be to selectively detachable from the frame 110. In some examples, each of the struts 112 include an engagement element at a distal portion thereof configured to engage the first portion 114A of the cover 114.

Exemplary engagement elements suitable for the present disclosure may include, but are not limited to snap-fit, grooves, projections, threads, latches, locking pins, magnets, and friction fit. For example, each strut 112 may include a hook or loop capable of extending through pores of the cover 114. In the example illustrated in FIGS. 1A-1B, the second portion 114B of the cover 114 is coupled to a distal portion of the sheath 102 via engagement elements 116 that secure the second portion 114B of the cover 114 to the sheath 102. The engagement elements 116 allow for the first portion 114A of the cover 114 to move (e.g., translate) relative to the second portion 114B without detaching the cover 114 from the sheath 102. In some examples, an inner surface of the sheath 102 proximate the distal end includes surface features to facilitate attachment of the cover 114. For example, the sheath 102 may include one or more projections or ridges configured to engage complementary features of the cover 114. FIG. 1C illustrates a top view of the retrieval device 106 while in the expanded configuration. As shown, distal portions of the four struts 112 of the frame 110 are coupled to the cover 114. A distal end of each of the struts 112 includes a loop that engages pores of the cover 114.

In an exemplary use of medical device 100, a user (e.g., medical professional) moves the retrieval device 106 distally using the handle 12 while the retrieval device 106 is in the collapsed configuration until the frame 110 advances distally beyond the distal end of the sheath 102. As the frame 110 moves distally, the frame 110 pulls the first portion 114A of the cover 114 outside the sheath 102 while the second portion 114B of the cover 114 remains coupled to the sheath 102. Once the frame 110 is unconstrained by the sheath 102, the frame 110 self-expands into the expanded configuration (FIG. 1B). In the expanded configuration, the user may capture a kidney stone or other object within the volume defined by the frame 110 and the cover 114.

Next, the user may insert the laser fiber 500 into the lumen 104 of the sheath 102 and advance the laser fiber 500 distally until it enters the volume defined by the expanded frame 110 and the cover 114, proximate the kidney stone captured within the volume. The user may operate the laser fiber 500 to break apart the kidney stone into smaller fragments and dust particles. The fragments may be retained by the cover 114, while the small dust particles pass through the cover 114 to be flushed via irrigation or the patient's urinary system.

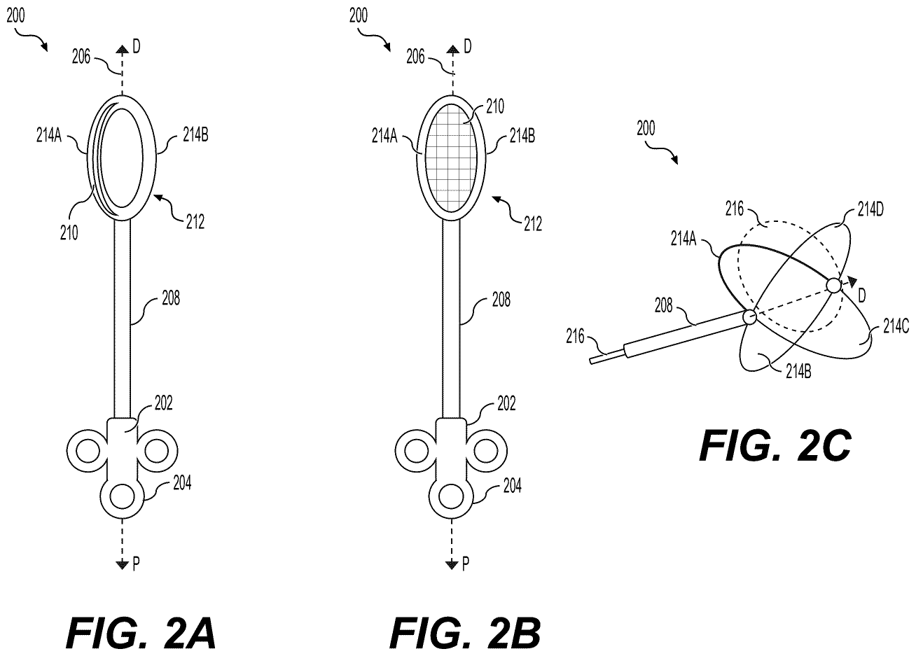

FIGS. 2A-2C illustrate another exemplary retrieval device 200 according to aspects of the present disclosure. The retrieval device 200 may include any of the features of retrieval device 106 unless otherwise specified herein. In this example, the retrieval device 200 includes a handle 202, a shaft 208, a frame 212 at a distal end of the shaft 208, and a cover 210 coupled to the frame 212. The retrieval device 200 may be used in combination with a sheath similar to sheath 102 of FIGS. 1A-1C, e.g., the retrieval device 200 being movable between a collapsed configuration within the sheath wherein the frame 212 has a reduced profile, and an expanded configuration (e.g., as shown in FIGS. 2A-2C) wherein the frame 212 has an expanded profile to define a volume for capturing objects within the body during use of the retrieval device 200. In such cases, the cover 210 is not attached to the sheath. The frame 212 may be self-expandable, e.g., comprising a self-expandable material such as Nitinol).

The cover 210 may be movable between a rolled configuration wherein the cover 210 extends along only a portion of the frame 212 and an unrolled configuration wherein the cover extends around the entire frame 212. For example, a user (e.g., medical professional) may transition the cover 210 between the rolled and unrolled configurations using the handle 202 as discussed further below.

The cover 210 may include any of the features of the cover 114 discussed above, e.g., the cover 210 being porous and configured to allow the passage of smaller particles, e.g., dust particles, while preventing passage of larger fragments. The cover 210 may comprise any of the laser-resistant materials discussed above in connection with the cover 114.

The frame 212 may include two or more struts extending radially outward from the shaft 208 relative to a central longitudinal axis 206 of the shaft 208. In the example shown in FIGS. 2A-2C, the frame 212 includes four struts 214A-214D, however it should be understood that the frame 212 may include more or fewer struts. The struts 214A-214D are coupled together at their proximal and distal ends. As mentioned above, the frame 212 may be self-expandable, e.g., the struts 214A-214D configured to transition from a collapsed configuration while positioned in a sheath, and an expanded configuration when unconstrained.

In some examples, the cover 210 is coupled to at least one of the struts 214A-214D. The cover 210 may include a first portion secured to a first strut 214A of the frame 212 with a second portion, e.g., a remainder, of the cover 210 movable between the rolled configuration and unrolled configuration. The cover 210 may be configured to unroll to engage with at least one of the other struts 214B-214D of the frame 212. In some examples, the cover 210 is configured to unroll around each of the other struts 214B-214D into the unrolled configuration, e.g., the cover 210 being coupled to each strut 214A-214D in the unrolled configuration. In some examples, the cover 210 may be movable between the unrolled and rolled configurations by rotating the shaft 208.

In some examples, the second portion of the cover 210 may be coupled to a wire 216 configured to facilitate transition between the rolled and unrolled configurations. The wire 216 may include a distal portion coupled to the cover 210, and a proximal portion coupled to one or more actuators of the handle 202, such that actuating the handle 202 causes the second portion of the cover 210 to move from the rolled configuration coupled to strut 214A into the unrolled configuration around each of the struts 214A-214D. In some examples, the handle 202 may include an actuator 204 configured to control the wire 224 to move the cover 210 between the rolled and unrolled configurations.

The cover 210 may be partially or completely concealed while in the rolled configuration. In some examples, the strut 214A to which the cover 210 is coupled in the rolled configuration may include a slot within the strut 214A. The first portion of the cover 210 may be coupled to the strut 214A within the slot, the second portion of the cover 210 being coupled to the wire 216 and capable of exiting the slot when pulled by the wire 216. In these examples, actuating the handle 202 causes the cover 210 to exit the slot of the strut 214A to transition from the rolled configuration to the unrolled configuration around each of the struts 214A-214D.

In some examples, at least one the struts 214B-214D is configured to engage a portion of the wire 216 to facilitate transition between the rolled and unrolled configurations. For example, one or more of the other struts 214B-214D may include surface features that guide the distal portion of the wire 216 around each of the struts 214B-214D to unroll the cover 210.

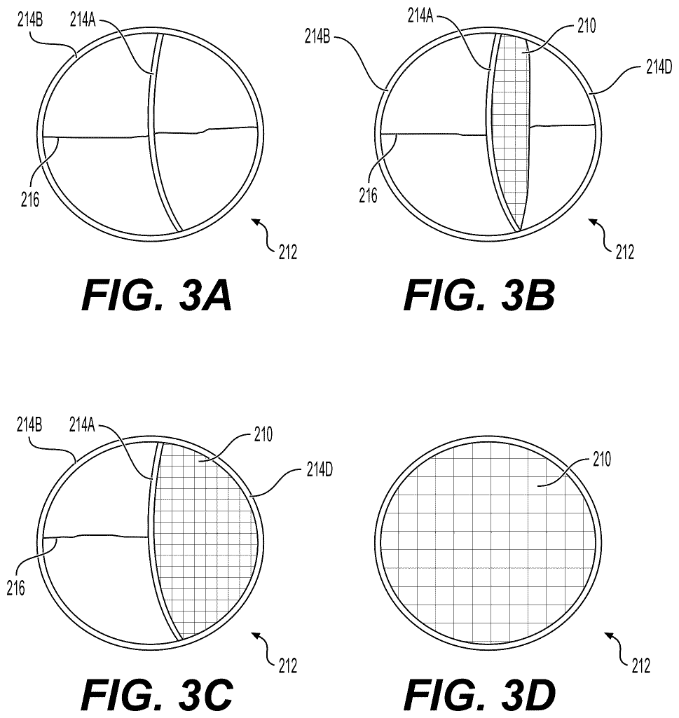

FIGS. 3A-3D illustrate different stages of deployment of the retrieval device 200, as the cover 210 transitions from the rolled configuration to the unrolled configuration. FIG. 3A shows the cover 210 in the rolled configuration coupled to strut 214A (e.g., concealed within a slot of the strut 214A). In this configuration, the frame 212 is expanded, such that a user may employ the retrieval device to capture kidney stones and/or other objects within the body. FIGS. 3B-3C show the cover 210 in-between the rolled configuration and the unrolled configuration, where FIG. 3B shows the cover 210 partially unrolled, FIG. 3C shows the cover 210 about 50% unrolled (e.g., covering about half of the expanded frame 212), and FIG. 3D shows the cover 210 in the fully unrolled configuration, e.g., around each of the struts 214A-214D. In the unrolled configuration, the user may be able to capture kidney stones and/or other objects and employ a laser fiber to reduce the size of the object(s) as discussed above in connection to FIGS. 1A-1C while the object(s) are captured within the expanded frame 212 and cover 210. The expanded frame 212 and cover 210 in the unrolled configuration may retain larger fragments while allowing small dust particles to pass through the cover 210 for removal from the body via irrigation or flushed through the body's urinary system.

While principles of this disclosure are described herein with reference to illustrative examples, it should be understood that the disclosure is not limited thereto. Those having ordinary skill in the art and access to the teachings provided herein will recognize additional modifications, embodiments, and substitution of equivalents all fall within the scope of the features described herein. Accordingly, the claimed features are not to be considered as limited by the foregoing description.

What is claimed is:

1. A medical device, comprising:
a sheath defining a lumen; and
a retrieval device slidably disposed within the lumen, the retrieval device including:
a shaft including an expandable frame at a distal end of the shaft; and
a cover coupled to the frame;
wherein the retrieval device is movable between a collapsed configuration while the frame is within the sheath and an expanded configuration while the frame is outside the sheath;
wherein the cover is porous and configured to prevent passage of particles greater than 5 mm in diameter therethrough; and
wherein each of the frame and the cover comprises a laser-resistant material.

2. The medical device of claim 1, wherein the frame is self-expandable.

3. The medical device of claim 1, wherein the frame comprises Nitinol.

4. The medical device of claim 1, wherein the cover comprises a laser-resistant polymer.

5. The medical device of claim 4, wherein the laser-resistant polymer of the cover comprises polypropylene, polyethylene, or a combination thereof.

6. The medical device of claim 4, wherein the laser-resistant polymer of the cover comprises high-density polyethylene, ultra-high-molecular-weight polyethylene, cross-linked polyethylene, or a combination thereof.

7. The medical device of claim 1, wherein pores of the cover are less than 1 mm in size.

8. The medical device of claim 1, wherein the sheath has a diameter ranging from about 2 mm to about 3 mm.

9. The medical device of claim 1, wherein the frame comprises a plurality of struts and the cover is coupled to each strut of the plurality of struts.

10. The medical device of claim 1, wherein the frame comprises a plurality of struts including at least one strut coupled to the cover and at least one strut unattached to the cover.

11. The medical device of claim 10, wherein the cover has a rolled configuration in the collapsed configuration, the cover being movable between the rolled configuration and an unrolled configuration in the expanded configuration of the retrieval device.

12. The medical device of claim 11, wherein the cover is movable between the rolled configuration and the unrolled configuration by rotating the shaft relative to the sheath.

13. The medical device of claim 10, wherein the plurality of struts includes at least four struts.

14. The medical device of claim 1, wherein the retrieval device is configured to allow passage of a laser fiber into a volume defined by the frame and the cover in the expanded configuration.

15. The medical device of claim 1, wherein a portion of the cover is coupled to the sheath.

16. A medical device, comprising:
a retrieval device including:
a shaft including an expandable frame at a distal end of the shaft, wherein the frame comprises a plurality of struts;
a cover coupled to the frame and movable between a rolled configuration and an unrolled configuration;
wherein the cover includes a laser-resistant material that is porous and configured to prevent passage of particles greater than 5 mm in diameter therethrough.

17. The medical device of claim 16, further comprising a sheath, wherein the retrieval device is slidably disposed with a lumen of the sheath.

18. The medical device of claim 17, wherein in the rolled configuration, the cover is coupled to fewer than all of the struts of the frame, and in the unrolled configuration, the cover is coupled to all of the struts of the frame.

19. A method comprising:
positioning a medical device proximate a target site within a body lumen of a patient, wherein the medical device comprises:
a sheath that defines a lumen; and
a retrieval device slidably disposed within the lumen, the retrieval including an expandable frame at a distal end of the shaft and a cover coupled to the frame;
moving the retrieval device distal to the sheath such that the frame transitions from a collapsed configuration within the sheath to an expanded configuration outside the sheath;
capturing an object at the target site within a volume defined by the frame and the cover in the expanded configuration of the retrieval device;
inserting a laser fiber through the sheath and into the volume; and
operating the laser fiber to break apart the object, wherein the cover comprises a laser-resistant material that is porous, such that the cover prevents passage of particles greater than 5 mm in diameter therethrough.

20. The method of claim 19, wherein operating the laser fiber generates dust particles less than or equal to 1 mm in size that pass through the cover.
